(19) United States
(12) Patent Application Publication
(10) Pub. No.: US 2009/0036839 A1
(43) Pub. Date: Feb. 5, 2009

(54) Ergonomic adaptor for repeated injections

(76) Inventor: Tom Phalen, Mount Hope (CA)

Correspondence Address: GOWLING, LAFLEUR HENDERSON LLP, ONE MAIN STREET WEST, HAMILTON, ON, L8P 4Z5, CA

(21) Appl. No.: 12/219,683

(22) Filed: Jul. 25, 2008

Related U.S. Application Data
(60) Provisional application No. 60/935,067, filed on Jul. 25, 2007.

(51) Int. Cl. A61M 5/315

(52) U.S. Cl. 604/227

(57) ABSTRACT

The present invention provides an adaptor for use with a fluid delivery device. The adaptor provides extended flanges to more easily accommodate a user's fingers and to disperse the counteracting pressure required when the plunger is depressed.

### FIELD OF INVENTION

The present invention relates to an adaptor for fluid delivery devices. The adaptor is designed to distribute the force required to force a plunger down a barrel and thereby to reduce the incidence of repetitive stress injuries.

### BACKGROUND OF THE INVENTION

Repetitive stress injuries comprise a group of conditions that result from overuse of a tool or prolonged repetition of a similar motion. One type of repeated motion that may lead to a repetitive stress injury is the delivery of fluids using a device, such as, but not limited to, a syringe. Health care workers are sometimes called upon to do many injections in a day. This can be particularly stressful when the injections are deep tissue injections that require a lot of pressure to be applied. It can also be a problem when the material to be injected is viscous or contains solids. Due to small flanges, a typical syringe requires a user to flex the first two fingers together.

Repetitive stress can also occur in other situations that involve repeated depression of a plunger. For example, in a veterinary situation where poultry or other animals are vaccinated, the healthcare professional may do hundreds or even thousands of injections in a relatively short period of time. In another example, laboratory workers may need to pipette many samples in a day for analytical, quantitative or diagnostic assays. Other manufacturing jobs may also require repeated depression of a plunger to transfer fluids.

There have been several attempts to relieve the stress associated with repeated use of fluid delivery devices. For example, automated medicament delivery systems comprising a hand-held gun-shaped device such as that described in United States patent application 2006/0247578 have been developed. However, these systems are expensive and have a limited range of depth of penetration. United States Patent application 2006/0270996 describes a fluid delivery apparatus that is activated by a scissors-like actuation mechanism. This device does reduce stress associated with repeated injections, but it is a fairly bulky device and may cause another type of stress since the actuation mechanism is always the same.

Thus, there remained a need for a device that can facilitate delivery of fluids by reducing the strain associated with repeated injections and by providing adaptability in the type of motion required.

### SUMMARY OF THE INVENTION

The present invention addresses the needs of the prior art by providing an adaptor for use with a fluid delivery device that dissipates the focus of pressure. The device is also adaptable in that it can be used in different ways thereby reducing the stress on muscles and joints due to repetitive movements.

According to an aspect of the present invention, there is provided an ergonomic adaptor for use with a fluid delivery device. The adaptor comprises a an elongated planar surface having a flange at each side and a bore in the middle. The flanges are typically at least twice the length of a flange at the top of a standard syringe.

In one preferred embodiment, the adaptor has a length of approximately 5-10 cm and a width of about 2 to 4 cm at the widest point.

In another preferred embodiment, the bore has a diameter in the range of about 1 to 4 cm. The size of the bore is adapted to the circumference of the barrel of the fluid delivery device. For example, the device may be adapted to fit over a 10 cc. syringe or a 50 cc. syringe, depending on the application.

In one preferred embodiment, the diameter of the bore varies from the top of the adaptor to the bottom of the adaptor so that a single adaptor can comfortably and snugly fit over barrels that vary in size and that have different circumferences.

In another embodiment, the diameter of the bore is constant along its length. Stop members are optionally included on the interior surface of the bore to adapt to barrels that vary in size.

In a further embodiment, the top of the bore includes a beveled edge.

In another preferred embodiment, the bore extends beyond the depth of the flanges to provide enhanced adaptability for a tight fit without excess bulk in the flanges.

### BRIEF DESCRIPTION OF THE DRAWINGS

These and other features of the invention will become more apparent from the following description in which reference is made to the appended drawings wherein:

FIG. 1 shows a perspective view of an adaptor in accordance with a first embodiment of the invention;

FIG. 2 shows a top view in accordance with the embodiment shown in FIG. 1;

FIG. 3 shows a side view in accordance with the embodiment shown in FIG. 1;

FIG. 4 illustrates diagrammatically how the adaptor can be fitted onto a syringe;

FIG. 5 shows an adaptor in combination with a syringe in accordance with a further embodiment of the present invention;

FIG. 6 shows the use of a conventional syringe;

FIG. 7 shows a use of an adaptor in accordance with an embodiment of the present invention;

FIG. 8 shows a use of the adaptor in accordance with a further embodiment of the present invention;

FIG. 9 shows a use of the adaptor in accordance with a further embodiment of the present invention; and

FIGS. 10 A to 10 D illustrate another embodiment of the invention where the adaptor is made of metal.

### DETAILED DESCRIPTION

To address the need to ameliorate the stress induced by multiple thumb depressions of a plunger, such as a syringe plunger, an ergonomic adaptor is provided. The adaptor is particularly useful in situations where a substantial amount of pressure needs to be applied, such as when repeated injections are done in deep muscles or when the fluid to be injected is viscous. The adaptor is also useful in other situations where the same movement is repeated many, many times in a short period. Situations such as this include, but are not limited to, mass vaccinations of humans or animals and mass laboratory or industrial transfer of fluids for analytical studies or for manufacturing.

To dissipate the forces required for fluid transfer and to reduce the incidence of the same movement over and over again, an adaptor is provided that increases the surface area used to depress a plunger. Because of the increased surface, the forces are spread out. In addition, the larger surface area allows a user to use different fingers and the palm of the hand to depress the plunger. This reduces the stress associated with repetition of the same movement, e.g. always using the thumb to depress and the two forefingers to counteract the force.

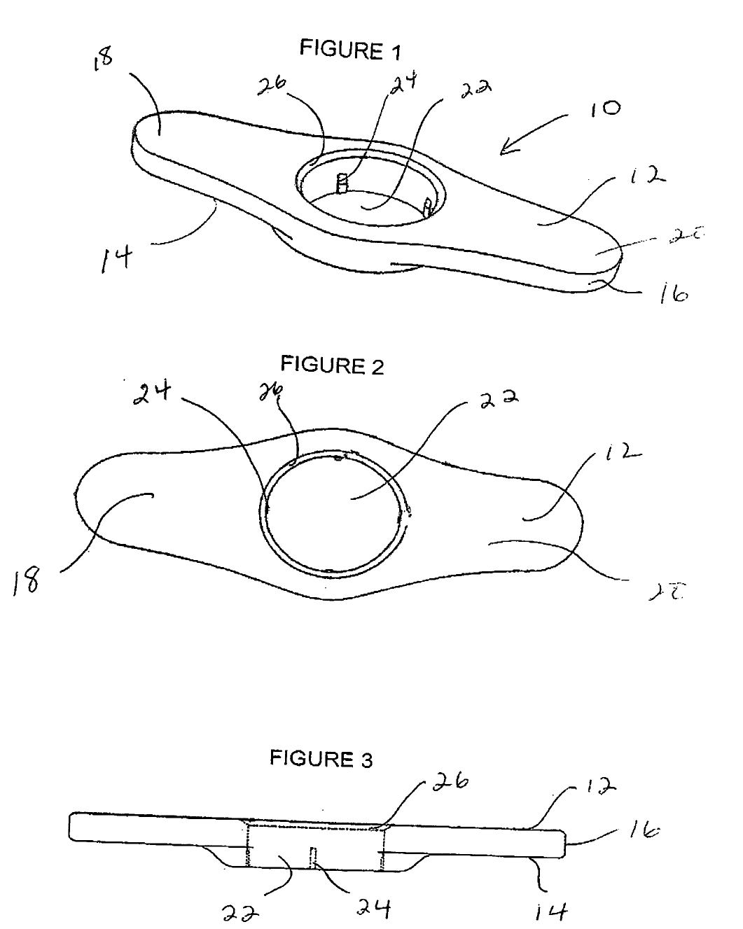

A more complete understanding of the invention can be obtained from the attached drawings showing preferred embodiments. Referring now to FIGS. 1 to 3, one embodiment of the adaptor is shown. The adaptor 10 comprises an upper surface 12 and a lower surface 14 with an adjoining wall 16. The two sides of the adaptor are contoured with round edges to form extended flanges 18, 20 and the middle of the adaptor includes a bore 22 that passes through it. The diameter of the bore may vary depending on the range of barrels it is designed to accommodate. The length of the bore may also vary. It may co-terminate with the lower surface of the adaptor or it may extend past the lower surface. Raised stops 24 are optionally located on the inside of the bore. The stops may be a constant size or they may be wedge shaped. The stops are provided to allow a firm fit even when there are slight variations in the size of the barrel. In a preferred embodiment, the upper surface 12 of the bore 22 includes a beveled edge 26. The beveled edge 26 allows the adaptor to engage the flanges of a typical syringe barrel to provide a secure fit.

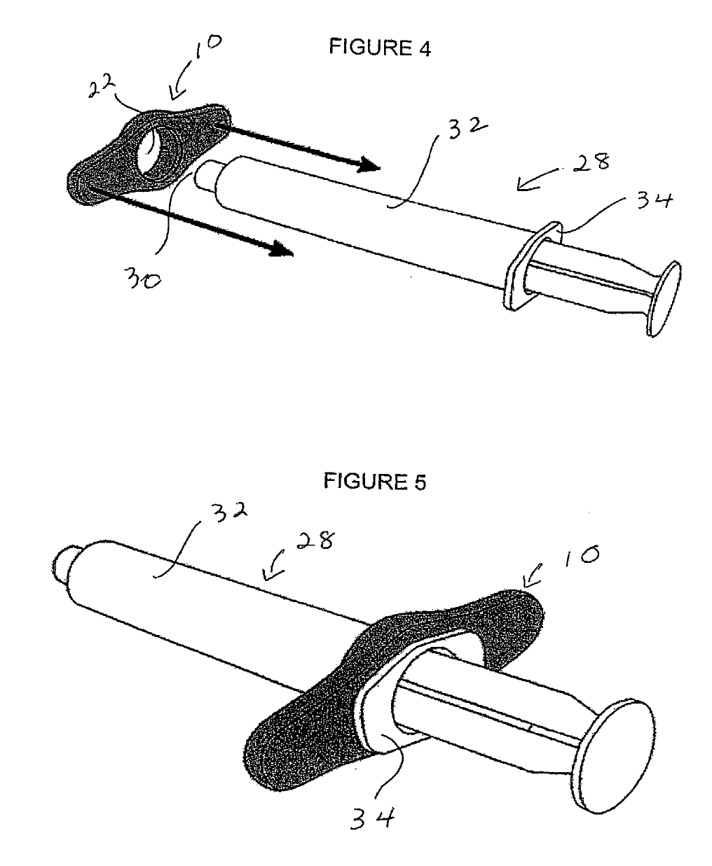

FIGS. 4 and 5 illustrate how the adaptor 10 can be applied to a typical syringe 28. The adaptor 10 is placed at the injection end 30 of the syringe and the barrel 32 of the syringe passes through the bore 22 in the adaptor 10. The adaptor 10 travels the length of the barrel 32 until it is stopped by the flanges 34 of the syringe. The adaptor fits snugly against these flanges and provides enlarged flanges for counteracting the forces applied to the plunger.

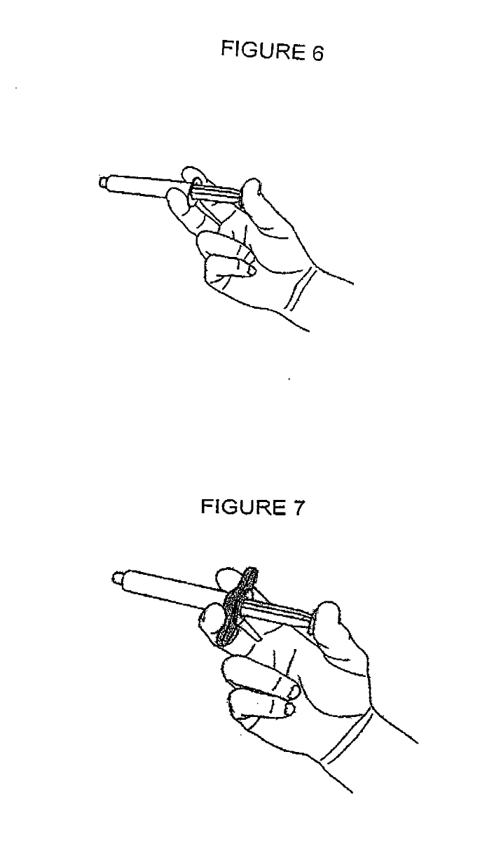

Different ways in which the adaptor can be used are shown in FIGS. 7 to 9. FIG. 6 shows a typical syringe in use. The flanges 34 on the syringe 28 barrel are small and barely accommodate a user's fingers. Thus a user must squeeze their fingers together and the surface area which absorbs the counteracting pressure is limited.

Referring now to FIG. 7, the syringe has been fitted with an adaptor 10 in accordance with the invention. The extended flanges 18, 20 of the adaptor provide a more comfortable means for holding the syringe. In addition, even when the typical forefingers and thumb grip is used, the extended surface area helps to dissipates the force required to deliver a fluid.

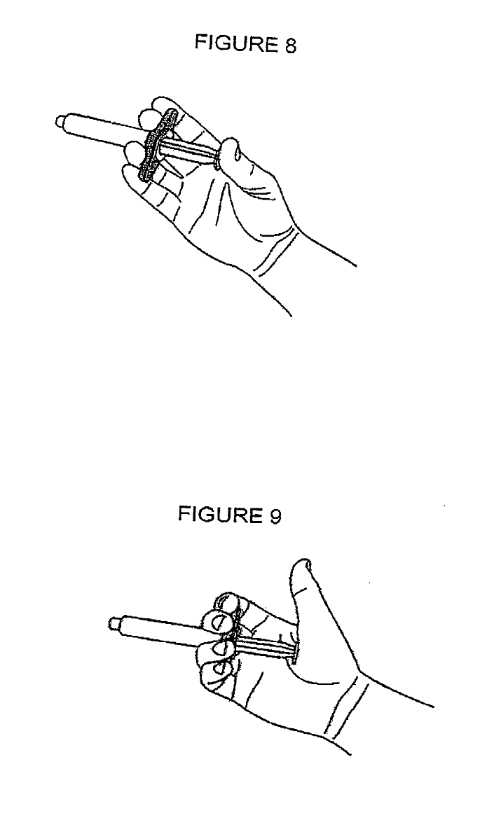

FIG. 8 illustrates another way in which the adaptor can be used. In this illustration, all four fingers are placed on the lower surface of the adaptor. It is apparent that four fingers can provide greater force than two fingers. It is not possible to use four fingers with a typical syringe. By using all four fingers the stress on any individual digit is reduced.

FIG. 9 shows yet another way in which the invention can be used. The syringe is placed between the two middle fingers and a portion of the palm is used to depress the plunger.

The adaptor of the present invention is constructed of a sturdy material that withstands the heavy forces sometime required to deliver a fluid through a syringe. It is preferably made of a washable, autoclavable material that can be used many times. Alternatively it may be provided as a disposable device. The adaptor provides options for a user to use different grips depending on the types and frequency of injections.

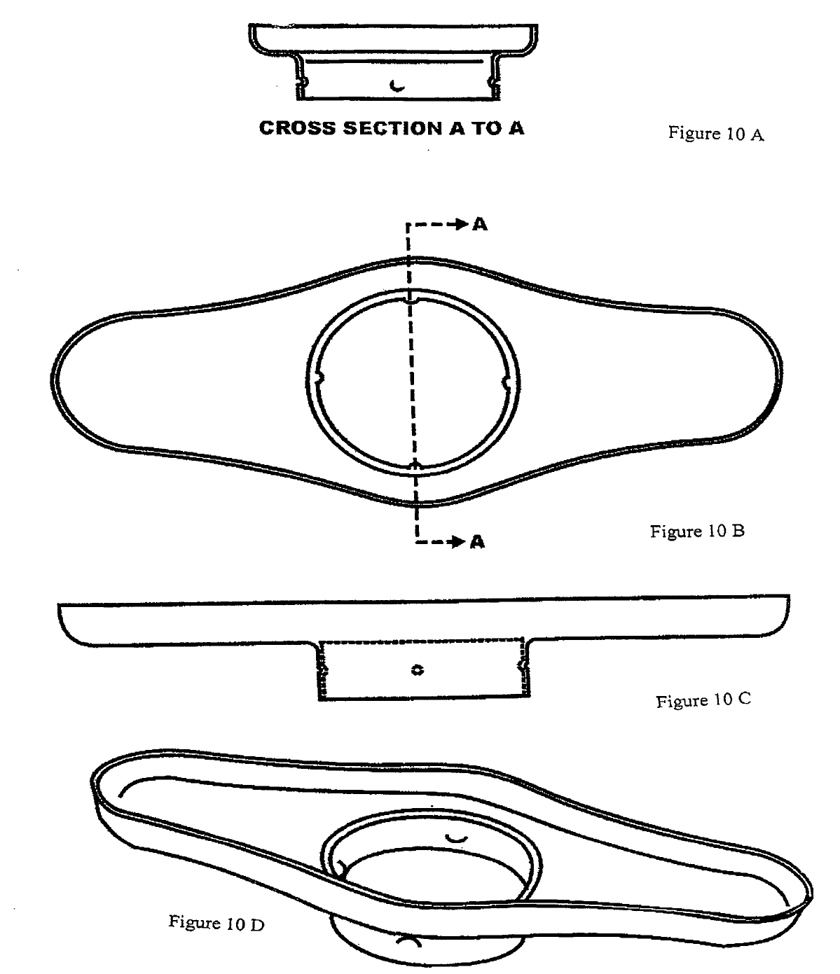

While it has been demonstrated that a plastic adaptor may be preferable in terms of cost and ease of manufacture, FIGS. 10A to 10 D demonstrate that a metal adaptor would be functionally equivalent.

It will be apparent to one skilled in the art that the material may be any material that withstands downward pressure. The present invention demonstrates the both plastic and metal adaptors can perform the same function/

While the adaptor has been illustrated in connection with a typical medical syringe, it is apparent that it can also be used with various other types of delivery devices that involve the depression of a plunger in a barrel, in particular, devices where the existing flanges are small and thus difficult to grip comfortably.

What is claimed is:

1. An adaptor for use with a fluid delivery device, said adaptor comprising:
a. an upper surface, a lower surface and a connecting wall; and
b. a bore located essentially in the middle of the adaptor and extending from the upper surface to the lower surface.

2. An adaptor according to claim 1 wherein the upper surface and the lower surface have elongated contoured edges that form flanges on either side of the bore.

3. An adaptor according to claim 1 wherein the adaptor has a width of about 2 to 4 cm at the centre.

4. An adaptor according to claim 3 wherein the width is about 2.5 cm.

5. An adaptor according to claim 1 having a length of about 5 to 10 cm.

6. An adaptor according to claim 5 wherein the length is about 6 to 7 cm.

7. An adaptor according to claim 1 wherein the bore has a diameter of about 1 to 4 cm.

8. An adaptor according to claim 7 wherein the bore has a diameter of about 1.6 cm.

9. An adaptor according to claim 7 wherein the bore has a depth of about 0.4 to 1 cm.

10. An adaptor according to claim 9 wherein the bore has a depth of about 0.7 cm.

11. An adaptor according to claim 1 wherein the bore includes an upper beveled edge.

12. An adaptor according to claim 1 further comprising a plurality of stop members affixed on the inner surface of the bore.

13. An adaptor according to claim 12 wherein the stop member is wedge shaped.
